(19) United States
(12) Patent Application Publication
(10) Pub. No.: US 2018/0235904 A1
(43) Pub. Date: Aug. 23, 2018

(54) CANNABIDIOL COMPOSITIONS INCLUDING MIXTURES AND USES THEREOF

(71) Applicant: EARTH SCIENCE TECH, INC., Boca Raton, FL (US)

(72) Inventors: Wei R. CHEN, Boca Raton, FL (US); Chad A. CURTIS, Hallandale, FL (US)

(73) Assignee: EARTH SCIENCE TECH, INC., Boca Raton, FL (US)

(21) Appl. No.: 15/517,915

(22) PCT Filed: Oct. 8, 2015
(86) PCT No.: PCT/US15/54768

§ 371 Date: Aug. 1, 2017

Related U.S. Application Data
(60) Provisional application No. 62/061,577, filed on Oct. 8, 2014.
(60) Provisional application No. 62/102,538, filed on Jan. 12, 2015.

(51) Int. Cl. A61K 31/05; A61K 31/015; A61K 31/122; A61K 36/185; A61P 35/00; A61N 5/06

(52) U.S. Cl. CPC A61K 31/05; A61K 31/015; A61K 31/122; A61N 2005/067; A61P 35/00; A61N 5/062; A61N 5/0625; A61K 36/185

(57) ABSTRACT

A pharmaceutical composition for use in the treatment of cancer is provided. The composition includes cannabidiol (CBD) and one or more of the following compounds: D-limonene, astaxanthin, malunggay, euphorbia hirta, pao extract, copaiba and ampalaya.

### RELATED APPLICATIONS

This application claims the benefit of U.S. Provisional Application No. 62/061,577, filed Oct. 8, 2014, and U.S. Provisional Application No. 62/102,538, filed Jan. 12, 2015, and PCT/US2015/054768, filed Oct. 8, 2015 the entire contents of each of which are incorporated herein by reference in its entirety.

### FIELD OF THE INVENTION

The present invention relates to compositions for treating cancer, modulating the immune system, and providing neuroprotection against reactive oxygen species and lipid peroxidation using naturally occurring compounds. More particularly, the invention relates to the use of compositions containing compounds from hemp oil in the treatment of cancer, the modulation of the immune system, and the neuroprotection against reactive oxygen species and lipid peroxidation.

### BACKGROUND OF THE INVENTION

### Cannabidiol

Cannabidiol (“CBD,” disclosed below) is one of at least 85 active cannabinoids identified in cannabis. It is a major phytocannabinoid, accounting for up to 40% of the plant's extract. CBD is considered to have a wider scope of medical applications than tetrahydrocannabinol (“THC”). An orally-administered liquid containing CBD has recently been used to treat dravet syndrome, a rare type of epilepsy. CBD's strong anti-oxidant properties are believed to have neuroprotective and anti-ischemic effects. CBD is also believed to minimize or counteract some of the pyschotomimetic effects of THC. Due to the natural source of CBD, study of it has been limited until relatively recently and its medical benefits and effects have not been adequately studied.

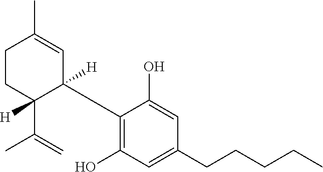 [SMILES: C=C(C)[C@@H]1CCC(C)=C[C@H]1c1c(O)cc(CCCCC)cc1O]

U.S. Pat. No. 6,630,507 titled “Cannabinoids as Antioxidants and Neuroprotectants,” owned by the U.S. Department Of Health And Human Services, postulates that CBD may be an effective antiepileptic, may lower intraocular pressure in the treatment of glaucoma, demonstrates neuro-protective properties, attenuates glutamate toxicity, protects against cellular damage, protects the brain from ischemic damage, and exhibits superior antioxidant activity which can be used in the prophylaxis and treatment of oxidation associated diseases. The patent states “Oxidative associated diseases include, without limitation, free radical associated diseases, such as ischemia, ischemic reperfusion injury, inflammatory diseases, systemic lupus erythematosus, myocardial ischemia or infarction, cerebrovascular accidents (such as a thromboembolic or hemorrhagic stroke) that can lead to ischemia or an infarct in the brain, operative ischemia, traumatic hemorrhage (for example a hypovolemic stroke that can lead to CNS hypoxia or anoxia), spinal cord trauma, Down's syndrome, Crohn's disease, autoimmune diseases (e.g., rheumatoid arthritis or diabetes), cataract formation, uveitis, emphysema, gastric ulcers, oxygen toxicity, neoplasia, undesired cellular apoptosis, radiation sickness, and others. The embodiments described in U.S. Pat. No. 6,630,507 are believed to be particularly beneficial in the treatment of oxidative associated diseases of the CNS, because of the ability of the cannabinoids to cross the blood brain barrier and exert their antioxidant effects in the brain. In particular embodiments of U.S. Pat. No. 6,630,507, the pharmaceutical composition of the present invention is used for preventing, arresting, or treating neurological damage in Parkinson's disease, Alzheimer's disease and HIV dementia; autoimmune neurodegeneration of the type that can occur in encephalitis, and hypoxic or anoxic neuronal damage that can result from apnea, respiratory arrest or cardiac arrest, and anoxia caused by drowning, brain surgery or trauma (such as concussion or spinal cord shock).

### D-Limonene

Limonene is a colorless liquid hydrocarbon classified as a cyclic terpene. The more common D-limonene isomer (disclosed below) possesses a strong smell of oranges. Its name is derived from lemon, and it is found in citrus fruit. It is used in chemical synthesis as a precursor to carvone and as a renewables-based solvent in cleaning products.

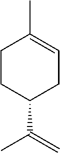

Limonene is common in cosmetic products. As the main odor constituent of citrus (plant family Rutaceae), D-limonene is used in food manufacturing and some medicines, e.g., as a flavoring to mask the bitter taste of alkaloids, and as a fragrant in perfumery; it is also used as botanical insecticide, particularly the (R)-(+)-enantiomer is most active as an insecticide. It is added to cleaning products such as hand cleansers to give a lemon-orange fragrance (see orange oil) and because of its ability to dissolve oils. In contrast, L-limonene has a piney, turpentine-like odor.

The principle metabolites of limonene are (+)- and (−)-trans-carveol, a product of 6-hydroxylation) and (+)- and (−)-perillyl alcohol, a product of 7-hydroxylation by CYP2C9 and CYP2C19 cytochromes in human liver microsomes. The enantiomers of perillyl alcohol have been investigated for their pharmacological activities as dietary chemotherapeutic agents. They are viewed as novel therapeutic options in some CNS neoplasms and other solid tumors, particularly for the treatment of gliomas. The cytotoxic activities of perillyl alcohol and limonene metabolites are likely due to their antiangiogenic activities, hyperthermia inducing effects as well as negative apoptosis regulation and Ras pathways. Limonene is an adenosine agonist which may explain its anti-stress and sedative properties.

D-limonene has been demonstrated to have chemopreventive and chemotherapeutic activity against many rodent solid tumor types. The chemopreventive activity of limonene during initiation can be attributed to the induction of phase I and phase II enzymes, with resulting carcinogen detoxification. The chemopreventive activity of limonene during promotion/progression may be due in part to inhibition of the posttranslational isoprenylation of growth-controlling small G proteins, such as p21ras. The complete regression of mammary carcinomas by limonene appears to involve tissue redifferentiation. The multiple antitumorigenic effects of limonene are attainable at a high therapeutic ratio, suggesting that limonene and related monoterpenes may be efficacious in the chemoprevention and chemotherapy of human malignancies.

D-limonene is one of the most common terpenes in nature. It is a major constituent in several citrus oils (orange, lemon, mandarin, lime, and grapefruit). D-limonene is listed in the Code of Federal Regulations as generally recognized as safe (GRAS) for a flavoring agent and can be found in common food items such as fruit juices, soft drinks, baked goods, ice cream, and pudding. D-limonene is considered to have fairly low toxicity. It has been tested for carcinogenicity in mice and rats. Although initial results demonstrated that d-limonene increased the incidence of renal tubular tumors in male rats, female rats and mice in both genders showed no evidence of any tumor. Subsequent studies have determined how these tumors occur and established that d-limonene does not pose a mutagenic, carcinogenic, or nephrotoxic risk to humans. In humans, d-limonene has demonstrated low toxicity after single and repeated dosing for up to one year. Being an excellent solvent for cholesterol, d-limonene has been used clinically to dissolve cholesterol-containing gallstones. Because of its gastric acid neutralizing effect and its support of normal peristalsis, it has also been used for relief of heartburn. D-limonene has well-established chemopreventive activity against many types of cancers. Evidence from a phase I clinical trial demonstrates a partial response in a patient with breast cancer and stable disease for more than six months in three patients with colorectal cancer.

### Astaxanthin

Astaxanthin (also known as Astaxanthine, Astaxantina, Dihydroxy-3,3′ dioxo-4,4′ bêta-carotène, Microalgae, Microalgue, Micro-Algue, Ovoester, 3,3′-dihydroxy-4,4′-diketo-beta-carotene, 3S,3′S-astaxanthin, 3R,3′R-astaxanthin, and 3R,3′S-astaxanthin) is an antioxidant terpene having the following formula:

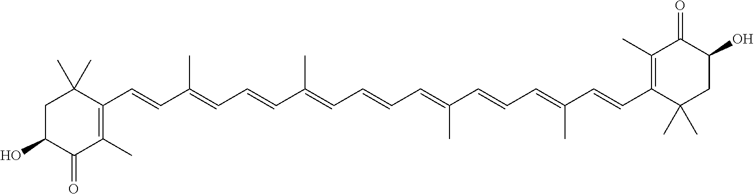 [SMILES: CC1=C(/C=C/C(C)=C/C=C/C(C)=C/C=C/C=C(C)/C=C/C=C(C)/C=C/C2=C(C)C(=O)[C@@H](O)CC2(C)C)C(C)(C)C[C@H](O)C1=O]

Astaxanthin is a carotenoid reddish pigment occurring naturally in certain algae, and is responsible for the pink or red color in salmon, trout, lobster, shrimp, and other seafood. Unlike several carotenes and one other known carotenoids, is not converted to vitamin A (retinol) in the human body. Like other carotenoids, astaxanthin has self-limited absorption orally and such low toxicity by mouth that no toxic syndrome is known. It is capable of crossing the blood-brain barrier, protects the brain and central nervous system, may lower cholesterol, increase lymphocytes, prevent heart disease, dementia, cancer, diabetes, liver disease, gastrointestinal disease, skin disease and male infertility.

Astaxanthin is used for treating Alzheimer's disease, Parkinson's disease, “brain attack” (stroke), high cholesterol, and an eye condition called age-related macular degeneration (AMD). It is also used for preventing cancer. The exact chemical pathways and the mechanisms of physiological effects are not entirely understood. It may also be beneficial in cardiovascular, immune, inflammatory and neurodegenerative diseases, and is applied directly to the skin for protection against sunburn.

### Malunggay

Malunggay (also known as Moringa Oleifera or moringa), is a plant found in tropical climates such as the Philippines, India and Africa. Malunggay is widely used as vegetable ingredient in cooking, as herbal medicine for a number of illness and other practical uses. It has a variety of uses, for example:

Antibacterial and anti-fungal uses. The Department of Biochemistry at the Indian Institute of Science in Bangalore (PLN Rao) found that malunggay leaves contain a compound “pterygospermin” that is known in medical science as having antimicrobial, antibacterial, anti-fungal properties.

Anti-Cancer. Malunggay or moringa has been shown in studies to have an anti-tumor capacity. Moringa contains benzyl isothiocyanate. Many studies show this chemical and its derivatives to have anti-cancer and chemoprotective capabilities. This chemoprotective ability helps strengthen cells so that they can tolerate chemotherapy. Malunggay is also considered in the treatment of prostate cancer and skin cancer.

Anti-inflammatory. Malunggay has been found to inhibit inflammation in a controlled scientific study conducted by Philippine DOST Scientists (Amelia P. Guevara, Carolyn Vargas and Milagros Uy). When an aqueous seed extract of malunggay is administered to a carrageenan induced inflammation, the aqueous seed extract of the Malunggay (Moringa Oleifera) inhibited the development of edema in rat-paw. Malunggay is traditionally used to prevent and treat inflammations associated with rheumatism, arthritis and joint pains.

In addition, preliminary studies show that malunggay may affect blood lipid profiles, although further clinical studies indicate it is not effective in human subjects. Antiasthmatic activity has also been attributed to finely powdered dried seed kernels.

### Euphorbia Hirta

Euphorbia hirta is widely used in traditional remedies and has been used cross-culturally for generations against maladies such as skin ailments, hypertension, female disorders, respiratory ailments (cough, coryza, bronchitis, and asthma), worm infestations in children, dysentery, jaundice, pimples, gonorrhea, digestive problems, and tumors. It contains a variety of organic compounds including alkanes, triterpenes, phytosterols, tannins, polyphenols, and flavanoids.

E. hirta is used in the treatment of gastrointestinal disorders (diarrhea, dysentery, intestinal parasitosis, etc.), bronchial and respiratory diseases (asthma, bronchitis, hay fever, etc.), and in conjunctivitis. Hypotensive and tonic properties are also reported in E. hirta. The aqueous extract exhibits anxiolytic, analgesic, antipyretic, and anti-inflammatory activities. The stem sap is used in the treatment of eyelid styes and a leaf poultice is used on swelling and boils.

Extracts of E. hirta have been found to show anticancer activity. The aqueous extract of the herb strongly reduced the release of prostaglandins I2, E2, and, D2. The aqueous extract also inhibits aflatoxin contamination in rice, wheat, maize, and mustard crops. Methanolic extract of leaves have antifungal and antibacterial activities. The leaves pounded with turmeric and coconut oil are warmed and rubbed on itchy soles. The latex of E. hirta is applied on lower eyelids, like surma to cure eye sores. The root exudate exhibits nematicidal activity against juveniles of meloidogyne incognita.

E. hirta herb shows antibacterial, anti-inflammatory, antimalarial, galactogenic, antiasthmatic, antidiarrheal, anticancer, antioxidant, antifertility, antiamoebic, and antifungal activities. Decoction of dry herbs is used for skin diseases. Decoction of fresh herbs is used as gargle for the treatment of thrush. Root decoction is also beneficial for nursing mothers deficient in milk. Roots are also used for snake bites. The polyphenolic extract of E. hirta has antiamoebic and antispasmodic activity. Quercitrin, a flavanoid glycoside, isolated from the herb showed an antidiarrheal activity. It is reported to have a relaxation effect on respiration. The alcoholic extract of whole plant shows hypoglycemic activity in rats. It has a sedative effect on the genitor-urinary tract.

Afzelin (I), quercitrin (II), and myricitrin (III) have been isolated from the methanolic extract of E. hirta. The chemical investigation of E. hirta has led to the isolation of rutin (IV), quercitin (V), euphorbin-A (VI), euphorbin-B (VII), euphorbin-C (VIII), euphorbin-D (IX), 2,4,6-tri-O-galloyl-β-D-glucose, 1,3,4,6-tetra-O-galloyl-β-D-glucose, kaempferol, gallic acid, and protocatechuic acid. E. hirta also contains β-amyrin, 24-methylenecycloartenol, β-sitosterol, heptacosane, nnonacosane, shikmic acid, tinyatoxin, choline, camphol, and quercitol derivatives containing rhamnose and chtolphenolic acid.

### Pao Pereira

Pao Pereira is an indigenous tree from the Amazon rainforest. The extract from the bitter inner-bark may suppress the proliferation of HIV, herpes viruses, cancer and leukemia cells. It may also be effective in preventing prostate cancer and/or reducing PSA (prostate specific antigen) levels. Pao Pereira has also been used in the treatment of malaria (Plasmodium falciparum) and to boost the immune system.

Alkaloids from Pao Pereira have demonstrated a toxic effect on certain cancer cells but little or no such effect on normal ones. Pao Pereira also may inhibit replication of the Herpes simplex virus genome. It crosses the blood-brain barrier easily and attaches itself to potential cancer cells. The alkaloids carry a risk of toxicity in cases of overdose but otherwise have no apparent side effects.

Many compounds have been isolated from Pao Pereira, including indole, beta-carboline alkaloids, geissospermine, flavopereirine, vello sine, geissoschizoline, geisso chizoline N4-oxide, 1,2-dehydrogeissoschizo line, apogeissoschizine, and pausperadine.

Pao extract, derived from bark of Amazonian tree Pao Pereira, is commonly used in South American medicine. A recent study showed that Pao extract repressed androgen-dependent LNCaP prostate cancer cell growth.

Pao extract suppressed CRPC PC3 cell growth in a dose- and time-dependent manner, through induction of apoptosis and cell cycle arrest. Pao extract treatment induced cell cycle inhibitors, p21 and p27, and repressed PCNA, Cyclin A and Cyclin D1. Pao extract also induced the upregulation of pro-apoptotic Bax, reduction of anti-apoptotic Bcl-2, Bcl-x_{L}, and XIAP expression, which were associated with the cleavage of PARP protein. Moreover, Pao extract treatment blocked PC3 cell migration and invasion.

Mechanistically, Pao extract suppressed phosphorylation levels of AKT and NFκB/p65, NFκB DNA binding activity, and luciferase reporter activity. Pao inhibited TNFα-induced relocation of NFκB/p65 to the nucleus, NFκB/p65 transcription activity, and MMP9 activity as shown by zymography. Consistently, NFκB/p65 downstream targets involved in proliferation (Cyclin D1), survival (Bcl-2, Bcl-x_{L}, and XIAP), and metastasis (VEGFa, MMP9, and GROα/CXCL1) were also downregulated by Pao extract. Finally, forced expression of NFκB/p65 reversed the growth inhibitory effect of Pao extract. Overall, Pao extract induced cell growth arrest, apoptosis, partially through inhibiting NFκB activation in prostate cancer cells. These data suggest that Pao extract may be beneficial for protection against CRPC.

### Ampalaya

Ampalaya, or Momordica charantia, has been used throughout Asia for many years for a variety of reasons. Different parts of the plant are used to relieve diabetes, as a stomachic, laxative, antibilious, emetic, anthelmintic agent, for the treatment of cough, respiratory diseases, skin diseases, wounds, ulcer, gout, and rheumatism. Ampalaya has a number of uses that are thought to be beneficial; including cancer prevention, treatment of diabetes, fever, HIV and AIDS, and infections.

In regards to the use of ampalaya for diabetes, several animal studies and small scale human studies have demonstrated a hypoglycemic effect of concentrated extracts. In addition, a 2014 review shows evidence that ampalaya, when consumed in raw or juice form, can be efficacious in lowering blood glucose levels. Contrary to this evidence, multiple reviews have found that ampalaya does not significantly decrease fasting blood glucose levels or A1c, indicators of blood glucose control, when taken in capsule or tablet form. While it may be beneficial in diabetes, the effects seem to depend on how it is consumed.

There is a continuous need to develop pharmaceutical compositions and methods for the treatment of cancer. In view of the beneficial properties of the foregoing natural compounds, it is desirable to develop pharmaceutical compositions that use these natural compounds in the treatment of cancer.

### The Brain

The brain contains trillions of cells. These cells include neurons with their long axons (nerve fibers) which can extend greater than 2 feet and serve to communicate to glial cells which support the brain in all its complex task. Two-thirds of the brain is composed of lipids. The vast majority of the different cell types in the brain require lipids for correct transmission of signals, providing energy and structural support to our cell membranes. Cells in the body and particularly in the brain are under constant attack by free radicals and oxidative stress. There are protective mechanisms that help deter this normal damage that occurs; however, with aging the protective mechanism declines substantially and degeneration of the brain occurs.

### Lipid Peroxidation

Lipid peroxidation is the degradation of lipids that occurs as a result of oxidative damage and is a useful marker for oxidative stress. Lipids are susceptible to an oxidative attack, typically by reactive oxygen species, resulting in a well-defined chain reaction with the production of end products such as malondialdehyde (MDA).

### Reactive Oxygen Species (ROS)

Reactive oxygen species are molecules that contain oxygen that are chemically active (i.e., oxygen ions and peroxides). They are formed as a natural byproduct of metabolism in our bodies. However, during stress and disease or dysfunction, these byproducts are not removed correctly and damage occurs in our body. The harmful effect of ROS occurs in cells in the form of DNA damage and oxidation of amino acids in proteins. ROS come in various forms such as superoxides and hydroxyl radicals and it is well documented that ROS play a crucial role in the formation of neurodegenerative diseases, cancer and aging.

In view of the foregoing, there is a need to develop pharmaceutical compositions and methods that provide a neuroprotective effect against the ravages of lipid peroxidation and ROS.

### SUMMARY OF THE INVENTION

One object of the present invention is to provide a pharmaceutical composition for use in the treatment of cancer that includes cannabidiol (CBD) and one or more of the following compounds: D-limonene, astaxanthin, malunggay, euphorbia hirta, pao pereira extract, copaiba and ampalaya.

Another object of the present invention is to provide a pharmaceutical composition for use in the treatment of cancer that includes hemp oil. The hemp oil is enriched with one or more of the following compounds: CBD, D-limonene, astaxanthin, malunggay, euphorbia hirta, pao pereira extract, copaiba and ampalaya.

Another object of the present invention is to provide a method of treating cancer (e.g., breast cancer). The method includes administering to a subject in need thereof an effective amount of a pharmaceutical composition that includes cannabidiol (CBD) and one or more of the following compounds: D-limonene, astaxanthin, malunggay, euphorbia hirta, pao pereira extract, copaiba and ampalaya.

Another object of the present invention is to provide a method of treating cancer (e.g., breast cancer). The method includes administering to a subject in need thereof an effective amount of a pharmaceutical composition that includes hemp oil. The hemp oil is enriched with one or more of the following compounds: CBD, D-limonene, astaxanthin, malunggay, euphorbia hirta, pao pereira extract, copaiba and ampalaya.

According to one embodiment, the pharmaceutical composition includes a pharmaceutically acceptable excipient.

According to another embodiment, the pharmaceutical composition is suitable for oral, parenteral, or topical administration.

According to another embodiment, the pharmaceutical composition includes D-limonene.

According to another embodiment, the pharmaceutical composition includes astaxanthin.

According to another embodiment, the pharmaceutical composition is used in a cancer treatment that includes one or more other cancer treatment methods.

According to another embodiment, the pharmaceutical composition is used in a cancer treatment that includes laser photothermal therapy.

Another object of the present invention is to provide a pharmaceutical composition for use in the treatment of cancer that includes hemp oil. The hemp oil is enriched with one or more of the following compounds: CBD, D-limonene, astaxanthin, malunggay, euphorbia hirta, pao pereira extract, copaiba and ampalaya.

According to another embodiment of the present invention, the concentration of hemp oil is greater than 10 μM and preferably greater than 25 μM.

According to another embodiment of the present invention, the concentration of CBD is greater than 1 μM, preferably greater than 2.5 μM and more preferably greater than 5.5 μM.

Another object of the present invention is to provide a method for activating the immune system. The method includes the stimulation of macrophages and the administration of the pharmaceutical compositions discussed above.

Another object of the present invention is to provide a method of modulating the immune system. The method includes the administration of the pharmaceutical compositions discussed above.

According to another embodiment of the present invention, the method of modulating the immune system includes incubating macrophages with the pharmaceutical compositions discussed above.

According to another embodiment of the present invention, the method of modulating the immune system includes inducing TNFα production.

Another object of the present invention is to provide a method for protecting human brain cells from reactive oxygen species and/or lipid peroxidation. The method includes the administration of the pharmaceutical compositions discussed above.

Another object of the present invention is to provide a method that prevents the death of human brain cells. The method includes the administration of the pharmaceutical compositions discussed above.

Another object of the present invention is to provide a method that provides neuroprotection against reactive oxygen species and lipid peroxidation. The method includes the administration of the pharmaceutical compositions discussed above.

The present invention utilizes hemp oil as a source of CBD and other components as a composition useful in treating or preventing cancer. Terpenes (e.g., D-limonene) may be used in the extraction process to create hemp oil and may be a component of the hemp oil, such as D-limonene.

Other aspects and advantages of the invention will be apparent from the following description and the appended claims.

### BRIEF DESCRIPTION OF THE DRAWINGS

FIG. 1: Chart of the viability of EMT6 tumor cells after incubation with hemp oil of different concentrations.

FIG. 2: Chart of the viability of 4T1 tumor cells and MEF normal cells after incubation with hemp oil of different concentrations.

FIG. 3: Chart of TNFα production by macrophages after incubation with hemp oil of different CBD concentrations.

FIG. 4: Chart of TNFα production by macrophages after incubation with LPS and hemp oil of different CBD concentrations.

FIG. 5: Earth Science Tech (ETST) High Grade CBD (Cannabidiol) Oil rescues human brain cells from reactive oxygen species in vitro. Human brain cells were cultured and treated with ETST High Grade CBD (Cannabidiol) Oil diluted to concentrations indicated on the graph for 2 days (1.0 μM CBD=8 ppm CBD rich hemp Oil; 2.5 μM CBD=20 ppm CBD rich hemp oil; 5.0 μM CBD rich hemp oil=41 ppm Hemp Oil). The cells were treated with 25 μM hydrogen peroxide (H₂O₂) for 16 hours and incubated with reactive oxygen species (ROS) master mix reagents for 1 hour to detect intracellular ROS. The amount of ROS was determined by fluorescence intensity at lex=490 nm/lem=525 nm. The experiment is performed in quadruplicates, with error bars representing standard deviation error. Control: DMSO only. Positive control: Media+H₂O₂+DMSO.

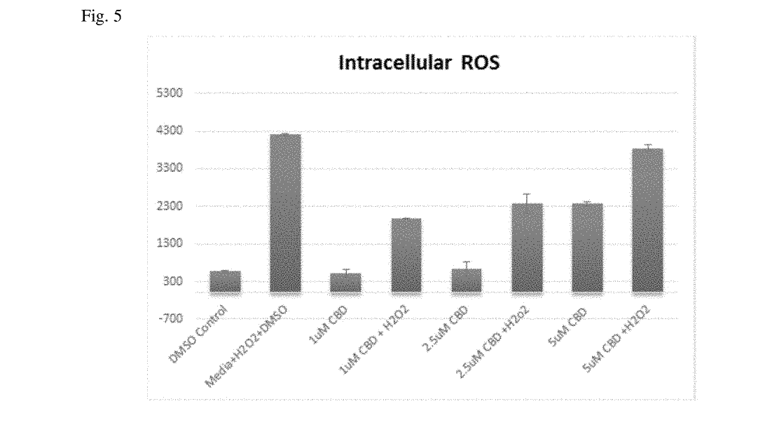

When brain cells are incubated with H₂O₂+DMSO intracellular ROS accumulates and is much higher than normal physiological states as seen in the figure. ETST Hemp CBD on its own maintains intracellular ROS to similar levels as seen in physiological states. When ETST Hemp CBD is used prophylactically at all concentrations used (1, 2.5 and 5 μM) there is a decrease in the formation of ROS.

FIG. 6: Earth Science Tech (ETST) High Grade CBD (Cannabidiol) Oil is protective against lipid peroxidation in human brain cells in vitro. Human brain cells were incubated with media (control), hydrogen peroxide (H₂O₂) to cause oxidative stress, DMSO which is the solvent to dilute ETST Hemp CBD in media and different concentrations of ETST Hemp CBD with and without H₂O₂. Error bar=Standard deviation. Control Media alone and DMSO alone. Positive control: H₂O₂.

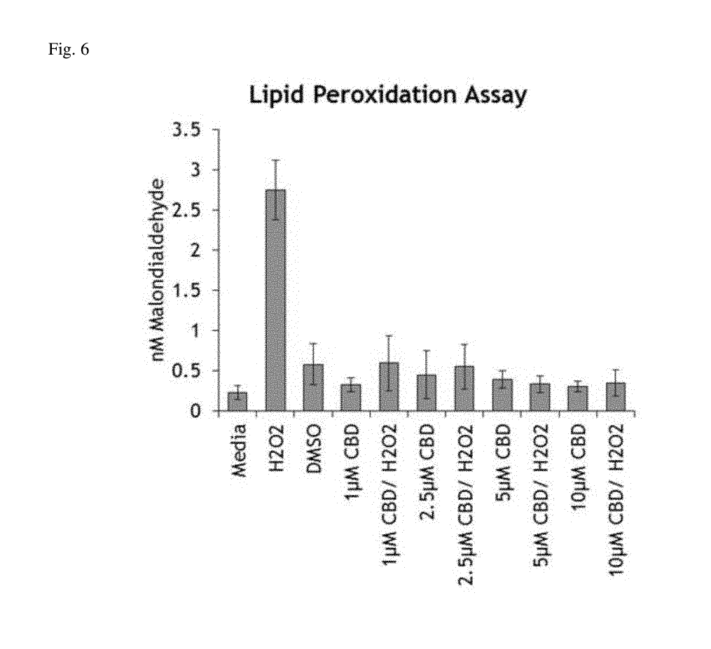

When brain cells are incubated with H₂O₂ by itself lipid peroxidation occurs substantially as seen in the graph above. Lipid peroxidation as measured my MDA present is seen to reach greater than 2.5 nM as compared to controls of media and DMSO alone which register 0.5 nM or less. When human brain cells are treated with ETST High Grade CBD (Cannabidiol) in concentrations varying from 1 μM to 10 μM prior to the addition of H₂O₂ (prophylactically), human brain cells are protected against the damaging effects of lipid peroxidation.

FIG. 7: Earth Science Tech (ETST) High Grade CBD (Cannabidiol) Oil deters cell death in human brain cells in vitro. Human brain cells were cultured and treated with ETST Hemp CBD diluted to concentrations indicated on the graph for 2 days (1.0 μM CBD=8 ppm CBD rich hemp oil; 2.5 μM CBD=20 ppm CBD rich hemp oil; 5.0 μM CBD=41 ppm CBD rich hemp oil). The cells were challenged with 25 μM (0.85 ppm) H₂O₂ for 15 hours and stained with neutral red, which stains only live cells. The amount of Neutral Red is quantified by absorbance at 540 nm. The experiment is performed in quadruplicates, with error bars representing standard deviations. Control: Media. Positive control: Media+DMSO+H₂O₂.

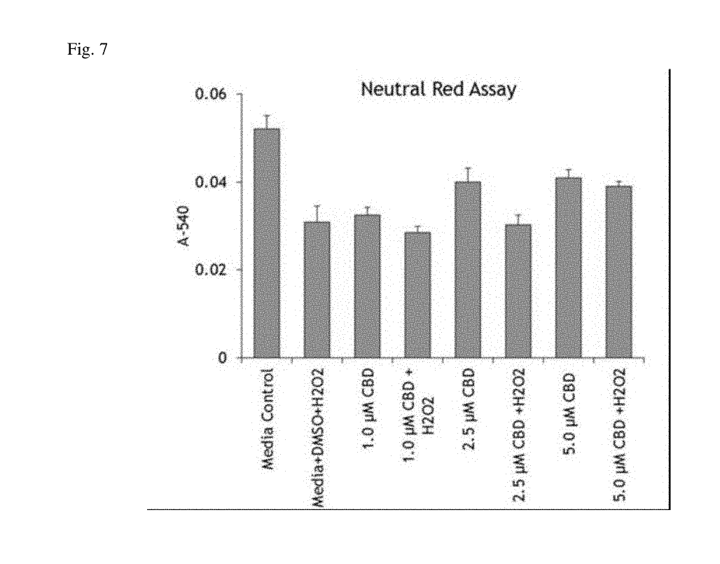

When human brain cells are incubated with H₂O₂ cell death occurs as compared to normal physiological states of growing cells as measured using neutral red assay. When ETST Hemp CBD is used prophylactically at various concentrations, cell death is deterred even in the presence of H₂O₂.

### DETAILED DESCRIPTION OF THE INVENTION

Hemp oil generally includes CBD in varying amounts depending upon how the hemp oil is prepared. As used herein, “hemp oil” generally refers to oil formed by leaching or extracting substantially nonaqueous compounds from the hemp plant. Those skilled in the art will appreciate that there are many methods of extracting oils and other hydrophobic or partially hydrophilic compounds from the hemp plant. The term “hemp oil” may generally refer to any of these oils, “extracts,” “essences,” “essential oils,” and may refer to any type of hemp oil, including “hash oil” and other oils that may include a variety of compounds found in hemp plants, also known as marijuana plants. Hemp and marijuana are well known as being a source for THC and other mind altering cannabinoids. However, for purposes of the present invention, the presence of THC and other chemical compounds are not necessary and may be undesirable. The hemp oil of the present invention may preferably include CBD, but the presence or absence of other compounds unique to the hemp plant are not relevant.

Hemp oil may be formed by leaching or extracting oils and/or hydrophobic compounds from hemp using any of a variety of techniques known in the art. Terpenes may be used to extract compounds and produce hemp oil. D-limonene, a terpene, may be used to form the hemp oil of the present invention. Alternatively, the enantiomer of D-limonene, limonene, may be used. It may also be desirable to use other terpenes in addition to, or in place of, D-limonene.

It may be desirable to increase the amount of CBD present in the hemp oil by enriching the hemp oil with CBD. In one embodiment, the invention comprises hemp oil enriched with CBD. Optionally, purified CBD may be dissolved or suspended or mixed with a solvent, or optionally a liquid which forms an emulsion or colloidal dispersion of CBD. Forming a CBD composition ab initio, without use of a hemp oil having multiple compounds extracted from hemp may be used instead of hemp oil. This may eliminate the inclusion of unwanted or unknown compounds. For example, CBD may be mixed with D-limonene to produce a suitable composition for use with the invention.

The pharmaceutical compositions may be used either alone or in combination with other treatments of cancer. These other treatments will depend on the type of cancer and can include but are not limited to surgery, chemotherapy, radiation therapy, immune therapy and laser photothermal therapy. These other treatments can be before, after or concurrent with the use of the pharmaceutical compositions of the present invention.

Pharmaceutical compositions suitable use in connection with the present invention are generally prepared by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, ordextrins; chelating agents such as EDTA; sugar alcohols such as mannitol or sorbitol; salt-forming counterions such as sodium; and/or nonionic surfactants such as TWEEN™, PLURONICS™ or PEG.

The formulations to be used for in vivo administration should generally be sterile. This is readily accomplished by filtration through sterile filtration membranes, prior to or following lyophilization and reconstitution.

Pharmaceutical compositions may take the form of any acceptable pharmaceutical formulation. Pharmaceutical compositions can be formulated in a variety of different forms, such as liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application.

The pharmaceutical compositions include those suitable for parenteral (including intravenous, subcutaneous, intradermal, intramuscular, and intraarticular), topical (including dermal, transdermal, transmucosal, buccal, sublingual, and intraocular), and rectal administration, although the most suitable route may depend upon, for example, the condition and disorder of the recipient.

Compositions for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The composition may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as EDTA, mannitol, 1,3-butanediol, water, Ringer's solution, an isotonic sodium chloride solution, or other suitable dispersing or wetting and suspending agents. The compositions may contain pharmaceutically acceptable substances or adjuvants, including, but not limited to, EDTA, e.g., 0.5 mM EDTA; pH adjusting and buffering agents and/or tonicity adjusting agents, e.g., sodium acetate, sodium lactate, sodium chloride, potassium chloride, calcium chloride, sorbitan monolaurate; minor amounts of non-toxic auxiliary substances, such as wetting or emulsifying agents or preservatives.

As used herein, the term “enriched” refers to the addition of some quantity of a substance to hemp oil. The hemp oil may or may not have had some of the substance being added before being enriched with that substance.

As used herein, the phrases “treating cancer” and “treatment of cancer” mean to inhibit the replication of cancer cells, inhibit the spread of cancer, decrease tumor size, lessen or reduce the number of cancerous cells in the body, or ameliorate or alleviate the symptoms of the disease caused by the cancer. The treatment is considered therapeutic if there is a decrease in mortality and/or morbidity, or a decrease in disease burden manifest by reduced numbers of malignant cells in the body.

A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition are outweighed by the therapeutically beneficial effects.

A “patient”, “subject” or “host” (these terms are used interchangeably) to be treated by the subject method may mean either a human or non-human animal.

The pharmaceutical compositions and methods of the present invention may be administered concurrently or sequentially with one or more other treatment methods. These methods include but are not limited to chemotherapy, radiation therapy, immune therapy, laser photothermal therapy and surgery.

The following examples are provided in order to demonstrate and further illustrate certain embodiments and aspects of the present invention and are not to be construed as limiting the scope thereof.

### Example 1

### Effects of Hemp Oil Mixtures on Animal Breast Cancer Cells and on Normal Cells

### Preparation of the CBD Solution

Raw material: Hemp oil, enhanced 6% CBD (386.57 D) with 150 mM CBD.

Hemp oil solution for experiment: 500 μl hemp oil dissolved in 1450 μl DMSO to achieve 5 mM CBD.

### Cell Lines

EMT6 is a non-metastatic mouse mammary cell line; 4T1 is a metastatic mouse mammary cell line; and MEF is mouse embryonic fibroblast cells used as normal cell for controls.

EMT6 cells and 4T1 cells were cultured in RPMI 1640 (GIBCO), and MEF cells were cultured in DMEM, supplemented with 15% fetal calf serum (FCS), penicillin (100 units/ml), and streptomycin (100 μg/ml) in 5% CO₂, 95% air at 37° C. in a humidified incubator.

### Cell Death Analysis

Cells were seeded in the 96 well (1,000 cell/well) with 100 μl medium in each well. After 12 hours, cells were incubated with hemp oil solution of different concentrations (0-50 μM) in cell samples (6 wells for each sample). Hemp oil solution was added daily with each change of medium for 4 days. The effects on cell proliferation were measured by MTS detection. OD492, the absorbance value at 492 nm, was read with a 96-well plate reader, to determine the viability of the cells.

### Results

### Dose Dependence of Growth Inhibition by Hemp Oil

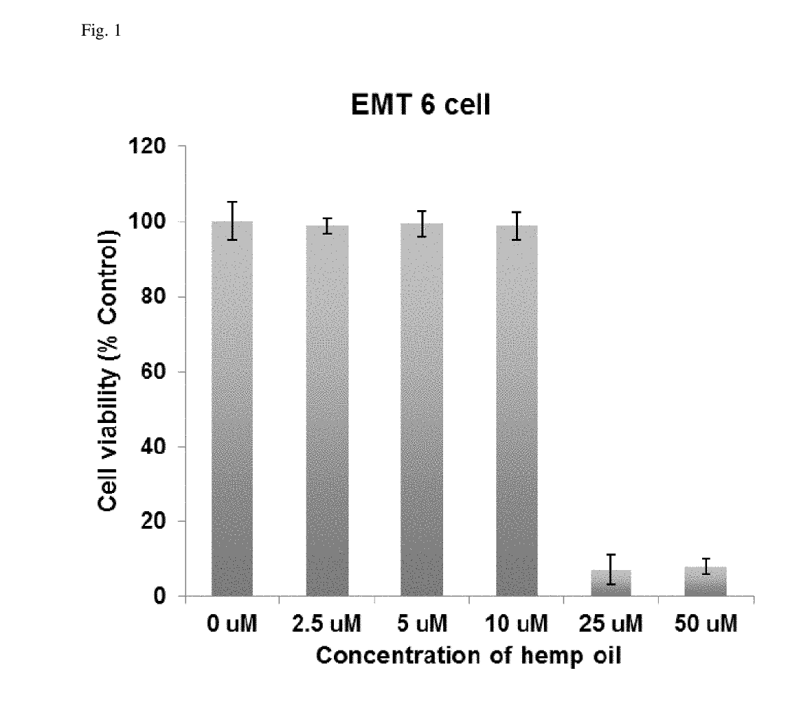

EMT6 breast cancer cell were incubated with hemp oil of different concentrations (0, 2.5, 5, 10, 25, 50 μM). Effects of hemp oils on EMT6 cells are given in FIG. 1.

FIG. 1 shows that the impact of hemp oil in the concentration range of 0 to 10 μM is not significant. Hemp oil with concentration higher than 25 μM shows a statistically significant cytotoxic effect, inhibited about 95% cell proliferation.

### Effects on Tumor and Normal Cells

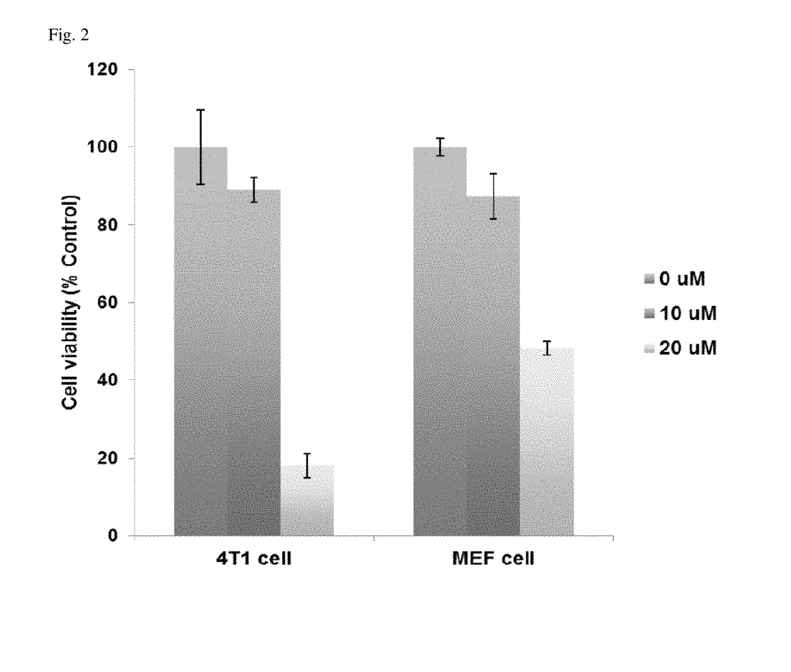

4T1 breast cancer cells and MEF normal cell were incubated with hemp oil with concentrations of 0, 10, and 20 μM, based on the effective doses obtained in FIG. 1. Effects of hemp oil on cell proliferation are shown in FIG. 2. FIG. 2 shows that at the effective dose of 20 μM, hemp oil inhibited the proliferation of cancer cells by nearly 90%, while only inhibited the proliferation of normal cells about 50%.

The results of this study indicate that with appropriate dose, in this case with 20 μM of hemp oil (6% CBD), helm oil can inhibit the proliferation of tumor cells more than that of normal cells.

### Example 2

### Effects of Hemp Oil Mixtures on Immune Cells

### Preparation of CBD Solution

The hemp oil (5.5% CBD) used in this experiment was provided by Earth Science Tech, Inc. The CBD solution was prepared by dissolving the hemp oil in DMSO solution to achieve appropriate CBD concentrations.

### Animal Immune Cell Line

RAW264.7, a mouse macrophage-like cell line, was used. The cells were cultured in DMEM, supplemented with 10% fetal calf serum (FCS), in 5% CO₂, 95% air, at 37° C. in an humidified incubator.

### Stimulation of Macrophages

The stimulation of macrophages was assessed by TNFα production, using a mouse TNFα ELISA kit.

To study the effects of hemp oil on macrophages, cells were seeded in a 24-well plate (2×105 cell/well) in 1-ml medium in each well for 12 h, and then incubated with hemp oil solution of different CBD concentrations (0-10 μM) for 24 h. Cell supernatant was collected for ELISA analysis.

To study the effects of hemp oil on enhancing macrophage stimulation by lipopolysaccharides (LPS), cells were seeded in a 24-well plate (2×105 cell/well) in 1-ml medium in each well for 12 h, and then incubated with LPS (1 ng/ml) and hemp oil solution of different CBD concentrations (0-10 μM) for 4 h. Cell supernatant was collected for ELISA analysis.

### Results

### Dose Dependence of Stimulation Effects on Macrophases by CBD

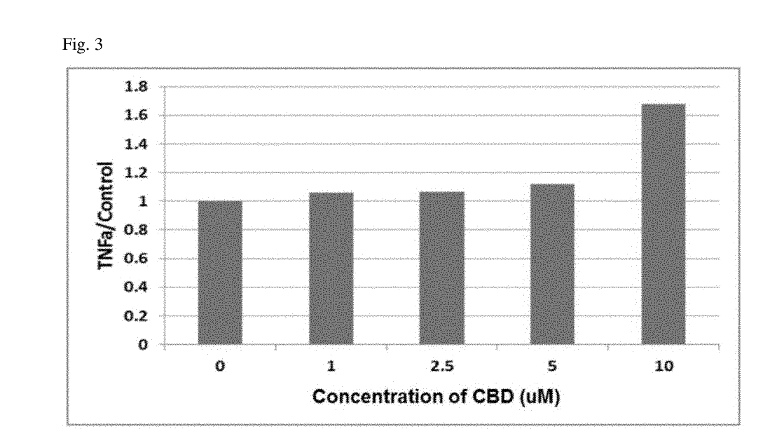

After the macrophages were incubated with hemp oil of different CBD concentrations (0, 1, 2.5, 5, 10 μM), the TNFα production by the cells was detected. The effects of CBD are given in FIG. 3.

The results in FIG. 3 indicate that the impact of CBD on macrophages in the concentration range of 0 to 5 μM is not significant. However, CBD with a concentration of 10 μM induced TNFα production at a significantly higher level.

### Effects of CBD on Enhancing LPS Induced TNFα Production

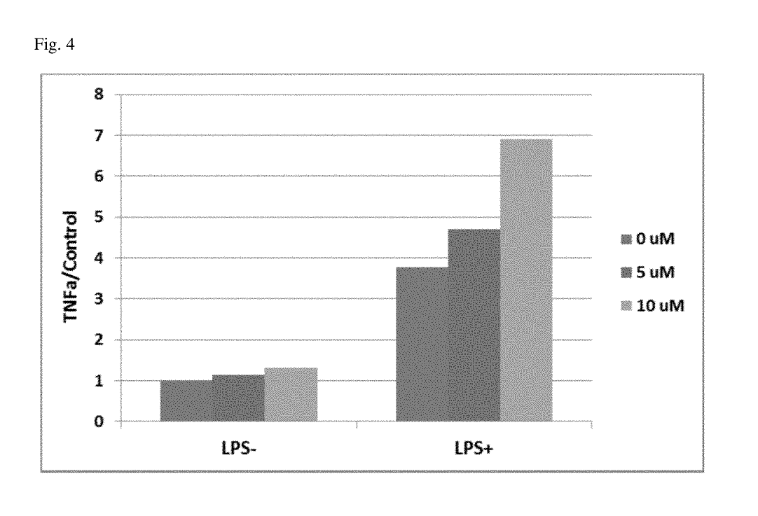

After macrophages were incubated with LPS (1 ng/ml) and hemp oil (CBD concentrations of 0, 5, and 10 μM) for 4 h, the TNFα production was detected. Effects of the LPS combined with hemp oil are shown in FIG. 4.

The results in FIG. 4 show that hemp oil can enhance TNFα production by macrophage stimulated with LPS, especially at a CBD dose of 10 μM.

### Conclusion

The results of this study indicate that with appropriate CBD concentrations, hemp oil can stimulate macrophages to produce TNFα (FIG. 3) and can also enhance the TNFα production stimulated by LPS (FIG. 4). Thus, the effects of hemp oil on immune cells may be used to improve cancer treatment.

Hemp oil mixtures containing CBD may also be mixed with other ingredients, such as, for example, astaxanthin, malunggay, euphorbia, Pao Pereira and ampalaya to further improve their beneficial qualities. For example, pao extract may be mixed with hemp oil and/or CBD mixtures to provide a treatment for various cancers. Similarly, methanolic and/or aqueous extracts of E. hirta may be mixed with hemp oil, CBD and pao extract, individually or in combination to enhance the immune boosting properties. In particular, pao extract and ampalaya may be used to provide a synergistic effect with CBD and/or hemp oil to treat cancer and slow metastasis. In addition, astaxanthin may be used to synergistically provide a toxic effect on cancer cells while not harming healthy cells. Ampalaya may be used to improve efficacy when treating diabetes or other blood sugar related illnesses.

### Example 3

### Fluorometric Intracellular ROS with a Kit that Provides a Sensitive, One-Step Fluorometric Assay to Detect Intracellular ROS (Especially Superoxide and Hydroxyl Radicals) after Induction of Peroxidation with Hydrogen Peroxide (With and without ETST High Grade Hemp CBD (Cannabidiol) Oil)

### Experimental Procedure ROS

1) Cells were plated in 96 well until it reach 90%. Neural cells were at a density of 2×10⁴ cells/cm². The cells were grown in neural media supplemented with 20 ng/ml of epidermal growth factor (EGF) and basic fibroblast growth factor (bFGF).
2) Media was changed every 2-3 days.
3) ETST Hemp CBD/Neural-Growth media was prepared by diluting the ETST Hemp CBD in dimethyl sulfoxide (DMSO). The suspension was filtered with a 0.22 micron filter. The ETST Hemp CBD suspension was then diluted in N-GRO media to contain the following concentrations of Hemp CBD: 5 μM, 2.5 μM, and 1 μM CBD.
4) Cells were incubated with ETST Hemp CBD for 48 hours prior to the addition of hydrogen peroxide.
4) After 48 hours, ROS was induced by addition of H₂O₂ at concentration of 25 μM to certain wells in quadruplicate. H₂O₂ was added to the wells of a 96 well plate according to the following design:

### 1) Set up the Master Reaction Mix.

2) Add 100 μL/well (96 well plate) Master Reaction Mix into the cell plate.
3) Incubate the cells in a 5% CO₂, 37° C. incubator for one hour.
4) Treat cells with 20 μL/well of H₂O₂ (96 well plate).
5) For control wells (untreated cells), PBS was used.
6) To induce ROS, incubate the cell plate in a 5% CO₂, 37° C. incubator for 16 hours.
7) Measure the fluorescence intensity at lex=490 nm/lem=525 nm for 1 second/well.

### Example 4

### Lipid peroxidation is Determined by the Reaction of MDA with Thiobarbituric Acid (TBA) to Form a Fluorometric Complex, Proportional to the Amount of MDA Present, after Induction of Peroxidation with Hydrogen Peroxide (With and without ETST High Grade Hemp CBD (Cannabidiol) Oil)

### Experimental Procedure Lipid Peroxidation

1) Two 24-well plates were coated with Matrigel for 1 hour at 27° C.
2) The plates were seeded with human neural cells at a density of 2×10⁴ cells/cm². The cells were grown in neural media supplemented with 20 ng/ml of epidermal growth factor (EGF) and basic fibroblast growth factor (bFGF).
3) Media was changed every 2-3 days.
4) ETST Hemp CBD/Neural-Growth media was prepared by diluting the ETST Hemp CBD in dimethyl sulfoxide (DMSO). The suspension was filtered with a 0.22 micron filter. The ETST Hemp CBD suspension was then diluted in N-GRO media to contain the following concentrations of Hemp CBD: 10 μM, 5 μM, 2.5 μM, and 1 μM CBD.
5) After the cells reached 80-90% confluency, base media was replaced with media containing ETST Hemp CBD or control media; the controls were media only, media with DMSO 1:100, and media with hydrogen peroxide (H₂O₂) (H₂O₂ was added during induction phase).
6) The cells were incubated under experimental conditions for 48 hours in media containing ETST Hemp CBD.
7) After 48 hours, lipid peroxidation was induced by addition of H₂O₂ at concentration of 100 μM to certain wells in duplicates. H₂O₂ was added according to the following protocol:

| | | | | | | |
|---|---|---|---|---|---|---|
| Well ID | A | B | C | D | E | F |

| | | | |
|---|---|---|---|
| 1 | Media only | 1 μM CBD + H₂O₂ | 5 μM CBD + H₂O₂ |
| | (control) | | |
| 2 | Media + DMSO | Media + 2.5 μM | Media + 10 μM |
| | (control) | CBD only | CBD only |
| 3 | Media + H₂O₂ | 2.5 μM CBD + H₂O₂ | 10 μM CBD + H₂O₂ |
| 4 | Media + 1 μM | Media + 5 μM | |
| | CBD only | CBD only | |

1) The cells were incubated with H₂O₂ for 120 minutes at 37° C.
2) Media was aspirated and the cells were lysed.
3) Total protein was precipitated, and reaction to form MDA-TBA adduct was performed. The reactions were placed in a 96-well plate.
4) MDA fluorometric detection was done by measuring fluorescence intensity (excitation 532 nm/emission 553 nm) of samples and standard curve.
5) Relative lipid peroxidation was measured by subtracting blank value from all readings.

### Example 5

### Neutral Red Assay to Determine Viability of Cells after Induction of Peroxidation with Hydrogen Peroxide (With and without ETST High Grade Hemp CBD (Cannabidiol) Oil)

### Experimental Procedure Neutral Red Assay

1) Human prenatal neural cells were seeded in a 24 well flask coated with Matrigel.
2) The cells were grown at 37° C., 90% humidity, and 5.0% CO₂ until they reach 80% confluency.
3) ETST Hemp CBD/Neural-Growth (N-GRO) media was prepared by diluting the ETST Hemp CBD in dimethyl sulfoxide (DMSO). The suspension was filtered with a 0.22 micron filter. The ETST Hemp CBD suspension was then diluted in N-GRO media to contain the following concentrations of Hemp CBD: 5 μM, 2.5 μM, and 1 μM CBD.
4) Cells were incubated with CBD for 48 hours prior to the addition of H₂O₂.
5) After 48 hours, H₂O₂ was added at concentration of 25 μM to certain wells in Duplicate. H₂O₂ was added according to the following protocol:
1) The cells were incubated with H₂O₂ for 16 hours at 37° C.
2) After incubation, media was aspirated from all wells and 100 uL of NR medium was added to each well.
3) Incubate for 1 hour with Neutral red (NR) medium at 37° C.
4) NR media was aspirated from all wells and washed each well with 150 μL PBS
5) 100 μl of lysis solution (ETOH/acetic acid) solution was added to all wells, including blanks.
6) Plate was shaken rapidly on a microtiter plate shaker for 20-45 min to extract NR.
7) Lysis solution was transferred to a 96-well plate and absorbance was determined at 540 nm in quadruplicate.

### Summary of Neuroprotective Effects

Reactive oxygen species (ROS) and lipid peroxidation is damaging to human brain cells. ETST High Grade CBD (Cannabidiol) Oil in varying concentrations has shown to rescue the damaging effects of ROS and lipid peroxidation, two oxidative stress pathways that play a crucial role in various neurodegenerative disorders, cancer and aging. Moreover, ETST High Grade CBD (Cannabidiol) Oil deters cell death that occurs following the addition of hydrogen peroxide, a known ROS and lipid peroxidation inducer. This suggests a neuroprotective effect in vitro to human brain cells following the use of ETST Hemp CBD. This data supports the use of ETST High Grade CBD (Cannabidiol) Oil in human clinical studies for neurodegenerative disorders such as Alzheimer's and Parkinson's.

### Compounds

The substances recited in the claims are commercially available and can be purchased from numerous suppliers. The following list provides possible suppliers of these substances and is in no way limiting.
CBD enriched hemp oil: Earth Science Tech, Inc. (Boca Raton, Fla.)
D-limonene: Parchem fine & specialty chemicals (New Rochelle, N.Y.)
Astaxanthin: Parchem fine &. specialty chemicals (New Rochelle, N.Y.)
Malunggay: Healthnest (Philippines)
Euphorbia hirta: TROPILAB® Inc. (St.Petersburg, Fla.)
Pao pereira extract: TROPILAB® Inc. (St. Petersburg, Fla.)
Copaiba: Edens Garden (San Clemente, Calif.)
Ampalaya: Go Natural (Philipines)

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

All references cited herein, including those below and including but not limited to all patents, patent applications, and non-patent literature referenced below or in other portions of the specification, are hereby incorporated by reference herein in their entirety.
1. U.S. Pat. No. 6,630,507.
2. Remington's Pharmaceutical Sciences 16th edition, Osol, A. Ed. (1980)

What is claimed is:

1. A pharmaceutical composition for use in the treatment of cancer, comprising:
cannabidiol (CBD); and
one or more compounds selected from the group consisting of D-limonene, astaxanthin, malunggay, euphorbia hirta, pao pereira extract, copaiba and ampalaya.

2. The pharmaceutical composition of claim 1, further comprising a pharmaceutically acceptable excipient.

3. The pharmaceutical composition of claim 1, wherein the pharmaceutical composition is suitable for oral, parenteral, or topical administration.

4. The pharmaceutical composition of claim 1, wherein the pharmaceutical composition comprises D-limonene.

5. The pharmaceutical composition of claim 1, wherein the pharmaceutical composition comprises astaxanthin.

6. The pharmaceutical composition of claim 1, wherein the pharmaceutical composition is used in a cancer treatment that comprises one or more other cancer treatment methods.

7. The pharmaceutical composition of claim 6, wherein the one or more other cancer treatment methods comprise laser photothermal therapy.

8. A pharmaceutical composition for use in the treatment of cancer, comprising:
hemp oil enriched with one or more compounds selected from the group consisting of cannabidiol (CBD), D-limonene, astaxanthin, malunggay, euphorbia hirta, pao pereira extract, copaiba and ampalaya.

9. The pharmaceutical composition of claim 8, wherein the pharmaceutical composition comprises CBD.

10. The pharmaceutical composition of claim 9, further comprising a pharmaceutically acceptable excipient.

11. The pharmaceutical composition of claim 9, wherein the pharmaceutical composition is suitable for oral, parenteral, or topical administration.

12. The pharmaceutical composition of claim 9, wherein the pharmaceutical composition further comprises D-limonene.

13. The pharmaceutical composition of claim 9, wherein the pharmaceutical composition further comprises astaxanthin.

14. The pharmaceutical composition of claim 9, wherein the pharmaceutical composition is used in a cancer treatment that comprises one or more other cancer treatment methods.

15. The pharmaceutical composition of claim 14, wherein the one or more other cancer treatment methods comprise laser photothermal therapy.

16. A method of treating breast cancer, comprising:
administering to a subject in need thereof an effective amount of the pharmaceutical composition of claim 1.

17. A method of treating breast cancer, comprising:
administering to a subject in need thereof an effective amount of the pharmaceutical composition of claim 4.

18. A method of treating breast cancer, comprising:
administering to a subject in need thereof an effective amount of the pharmaceutical composition of claim 5.

19. A method of treating breast cancer, comprising:
administering to a subject in need thereof an effective amount of the pharmaceutical composition of claim 8.

20. A method of treating breast cancer, comprising:
administering to a subject in need thereof an effective amount of the pharmaceutical composition of claim 9.

21. A method of modulating the immune system, comprising:
administering to a subject in need thereof an effective amount of the pharmaceutical composition of claim 1.

22. A method of modulating the immune system, comprising:
administering to a subject in need thereof an effective amount of the pharmaceutical composition of claim 4.

23. A method of modulating the immune system, comprising:
administering to a subject in need thereof an effective amount of the pharmaceutical composition of claim 5.

24. A method of modulating the immune system, comprising:
administering to a subject in need thereof an effective amount of the pharmaceutical composition of claim 8.

25. A method of modulating the immune system, comprising:
administering to a subject in need thereof an effective amount of the pharmaceutical composition of claim 9.

26. A method of providing neuroprotection against reactive oxygen species and lipid peroxidation, comprising:
administering to a subject in need thereof an effective amount of the pharmaceutical composition of claim 1.

27. A method of providing neuroprotection against reactive oxygen species and lipid peroxidation, comprising:
administering to a subject in need thereof an effective amount of the pharmaceutical composition of claim 4.

28. A method of providing neuroprotection against reactive oxygen species and lipid peroxidation, comprising:
administering to a subject in need thereof an effective amount of the pharmaceutical composition of claim 5.

29. A method of providing neuroprotection against reactive oxygen species and lipid peroxidation, comprising:
administering to a subject in need thereof an effective amount of the pharmaceutical composition of claim 8.

30. A method of providing neuroprotection against reactive oxygen species and lipid peroxidation, comprising:
administering to a subject in need thereof an effective amount of the pharmaceutical composition of claim 9.
